(19) United States
(12) Patent Application Publication
(10) Pub. No.: US 2026/0325414 A1
(43) Pub. Date: Oct. 22, 2026

(54) COSMETIC COMPOSITION COMPRISING SELECTED OIL AND DIOL

(71) Applicant: L'OREAL, PARIS (FR)

(72) Inventors: Yusuke IIMA, Kawasaki-shi (JP); Nahoko NAKASHIMA, Kawasaki-shi (JP); Romain TACHON, Kawasaki-shi (JP); Mina MUTO, Kawasaki-shi (JP)

(21) Appl. No.: 18/880,943

(22) PCT Filed: Jun. 23, 2023
(86) PCT No.: PCT/JP2023/024301

§ 371 Date: Jan. 3, 2025

(30) Foreign Application Priority Data
Jul. 12, 2022 (JP) 2022-111769
Aug. 18, 2022 (FR) 2208357

(51) Int. Cl. A61K 8/06; A61K 8/34; A61K 8/37; A61Q 19/00

(52) U.S. Cl. CPC A61K 8/062; A61K 8/342; A61K 8/345; A61K 8/37; A61Q 19/00; A61K 2800/591

(57) ABSTRACT

A composition contains: (a) at least one oil; (b) at least one diol having at least one carbon chain comprising 3 or more carbon atoms, with the total number of carbon atoms of 5 or 6; and (c) water. The (a) oil is selected from the group of (i) fatty alcohols, (ii) fatty acids, (iii) monovalent carboxylic acid monoesters, (iv) divalent carboxylic acid diesters, (v) N-acyl amino acid esters, and (vi) alkyl lactates, under specific conditions. The weight ratio of the amount of the (b) diol having at least one carbon chain comprising 3 or more carbon atoms, with the total number of carbon atoms of 5 or 6 to the amount of the (a) oil is 4 or more, preferably 5 or more, and more preferably 6 or more. The composition is stable over time and can show a transparent or slightly translucent aspect.

### TECHNICAL FIELD

The present invention relates to a composition, preferably a cosmetic composition, and more preferably a skin cosmetic composition.

### BACKGROUND ART

Oil-in-water (O/W) and water-in-oil (W/O) emulsions are well known in the field of cosmetics and dermatology, in particular for the preparation of cosmetic products, such as milks, creams, tonics, serums and lotions. In particular, a fine emulsion such as an O/W nano- or micro-emulsion is particularly interesting due to its transparent or slightly translucent aspect.

Typically, fine emulsions can be formed by using a surfactant. For example, WO 2014/98265 discloses a nano- or micro-emulsion which is formed by using a combination of some surfactants.

### DISCLOSURE OF INVENTION

However, fine emulsions are sometimes unstable over time, in particular under temperature changes, e.g., changes from hot to cool temperatures and vice versa, such that they cause phase separation, and do not show a transparent or slightly translucent aspect.

An objective of the present invention is to provide a composition which is stable over time and can show a transparent or slightly translucent aspect.

The above objective of the present invention can be achieved with a composition comprising:
(a) at least one oil;
(b) at least one diol having at least one carbon chain comprising 3 or more carbon atoms, with the total number of carbon atoms of 5 or 6; and
(c) water,
wherein
the (a) oil is selected from the group consisting of (i) fatty alcohols, (ii) fatty acids, (iii) monovalent carboxylic acid monoesters, (iv) divalent carboxylic acid diesters, (v) N-acyl amino acid esters, and (vi) alkyl lactates, provided that
   the (i) fatty alcohols and the (ii) fatty acids comprise a branched alkyl group or a linear or branched alkenyl group, each having 4 to 30, preferably 5 to 26, and more preferably 6 to 22 carbon atoms,
   the (iii) monovalent carboxylic acid monoesters comprise a branched alkyl group or a linear or branched alkenyl group, each having 4 to 30, preferably 5 to 26, and more preferably 6 to 22 carbon atoms,
   the (iv) divalent carboxylic acid diesters comprise at least one branched alkyl group having 3 to 10, preferably 3 to 8, and more preferably 3 to 6 carbon atoms, and an alkylene group having 6 to 18, preferably 6 to 14, and more preferably 6 to 10 carbon atoms,
   the (v) N-acyl amino acid esters comprise a branched alkyl group having 3 to 10, preferably 3 to 8, and more preferably 3 to 6 carbon atoms, and a linear or branched acyl group having 4 to 30, preferably 5 to 26, and more preferably 6 to 22 carbon atoms, and
   the (vi) alkyl lactates comprise a linear or branched alkyl group or a linear or branched alkenyl group, each having 4 to 30, preferably 5 to 26, and more preferably 6 to 22 carbon atoms,
and
the weight ratio of the amount of the (b) diol having at least one carbon chain comprising 3 or more carbon atoms, with the total number of carbon atoms of 5 or 6, to the amount of the (a) oil is 4 or more, preferably 5 or more, and more preferably 6 or more.

The (i) fatty alcohol may be represented by the following general formula (1):

R¹—OH (1)
wherein
R¹ represents a branched alkyl group, or a linear or branched alkenyl group, each having 4 to 30, preferably 5 to 26, and more preferably 6 to 22 carbon atoms. It is preferable that the (i) fatty alcohol be selected from the group consisting of isostearyl alcohol, oleyl alcohol, and a mixture thereof.

The (ii) fatty acid may be represented by the following general formula (2):

R¹—COOH (2)
wherein
R¹ represents a branched alkyl group, or a linear or branched alkenyl group, each having 4 to 30, preferably 5 to 26, and more preferably 6 to 22 carbon atoms. It is preferable that the (ii) fatty acid be selected from the group consisting of isostearic acid, oleic acid, and a mixture thereof.

The (iii) monovalent carboxylic acid monoester may be represented by the following general formula (3):

R²—COO—R³ (3)
wherein
R² represents a branched alkyl group or a linear or branched alkenyl group, each having 4 to 30, preferably 5 to 26, and more preferably 6 to 22 carbon atoms, and
R³ represents a branched alkyl group having 3 to 10, preferably 3 to 8, and more preferably 3 to 6 carbon atoms. It is preferable that the (iii) monovalent carboxylic acid monoester be isopropyl isostearate.

The (iv) divalent carboxylic acid diester may be represented by the following general formula (4):

R²—OC(═O)—R⁴—C(═O)O—R³ (4)
wherein
R² and R³ independently represent a branched alkyl group having 3 to 10, preferably 3 to 8, and more preferably 3 to 6 carbon atoms, and
R⁴ represents a linear or branched alkylene group having 6 to 18, preferably 6 to 14, and more preferably 6 to 10 carbon atoms. It is preferable that the (iv) divalent carboxylic acid diester be selected from the group consisting of diisopropyl adipate, diisopropyl sebacate, and a mixture thereof.

The (v) N-acyl amino acid ester may be represented by the following general formula (5):

R²—N(R⁵)—CH(R⁶)—(CH₂)ₙ—COO—R³ (5)
wherein
R² represents a linear or branched, acyl group, having 4 to 30, preferably 5 to 26, and more preferably 6 to 22 carbon atoms,
R³ represents a branched alkyl group having 3 to 10, preferably 3 to 8, and more preferably 3 to 6 carbon atoms,
R⁵ represents a hydrogen atom, or a linear or branched alkyl group having 1 to 4 carbon atoms,
R⁶ represents a hydrogen atom, or a linear or branched alkyl group having 1 to 4 carbon atoms which may be substituted with a hydroxy group or a phenyl group, and
n represents an integer from 0 to 2. It is preferable that the (v) N-acyl amino acid ester be isopropyl lauroyl sarcosinate.

The (vi) alkyl lactate may be represented by the following general formula (6):

CH₃—C(OH)—COO—R⁷ (6)
wherein
R⁷ represents a linear or branched alkyl group or a linear or branched alkenyl group, each having 4 to 30, preferably 5 to 26, and more preferably 6 to 22 carbon atoms. It is preferable that the (vi) alkyl lactate be selected from the group consisting of lauryl lactate, isostearyl lactate, and a mixture thereof.

The amount of the (a) oil(s) in the composition according to the present invention may range from 0.5% to 25% by weight, preferably from 1% to 20% by weight, and more preferably from 2% to 15% by weight, relative to the total weight of the composition.

The (b) diol having at least one carbon chain comprising 3 or more carbon atoms, with the total number of carbon atoms of 5 or 6, may be selected from the group consisting of dipropylene glycol, pentylene glycol, hexylene glycol, and a mixture thereof.

The composition according to the present invention may further comprise (d) at least one monovalent alcohol, preferably a C₂₋₄ aliphatic monovalent alcohol, and more preferably ethanol. In this case, the weight ratio of the total amount of the (b) diol(s) having at least one carbon chain comprising 3 or more carbon atoms, with the total number of carbon atoms of 5 or 6 and the (d) monovalent alcohol(s) to the amount of the (a) oil may be 4 or more, preferably 5 or more, and more preferably 6 or more.

The composition according to the present invention may have a turbidity of 120 NTU or less, preferably 90 NTU or less and more preferably 60 NTU or less.

The composition according to the present invention may be a cosmetic composition, preferably a skin cosmetic composition, and more preferably a skin care cosmetic composition.

The present invention also relates to a cosmetic process for treating a keratin substance, preferably skin, comprising the step of applying the composition according to the present invention.

### BEST MODE FOR CARRYING OUT THE INVENTION

After diligent research, the inventors have discovered that it is possible to provide a composition which is stable over time and can show a transparent or slightly translucent aspect, by using a combination of selected oil and diol.

Thus, the present invention relates to a composition comprising:
(a) at least one oil;
(b) at least one diol having at least one carbon chain comprising 3 or more carbon atoms, with the total number of carbon atoms of 5 or 6; and
(c) water,
wherein
the (a) oil is selected from the group consisting of (i) fatty alcohols, (ii) fatty acids, (iii) monovalent carboxylic acid monoesters, (iv) divalent carboxylic acid diesters, (v) N-acyl amino acid esters, and (vi) alkyl lactates, provided that
   the (i) fatty alcohols and the (ii) fatty acids comprise a branched alkyl group or a linear or branched alkenyl group, each having 4 to 30, preferably 5 to 26, and more preferably 6 to 22 carbon atoms,
   the (iii) monovalent carboxylic acid monoesters comprise a branched alkyl group or a linear or branched alkenyl group, each having 4 to 30, preferably 5 to 26, and more preferably 6 to 22 carbon atoms,
   the (iv) divalent carboxylic acid diesters comprise at least one branched alkyl group having 3 to 10, preferably 3 to 8, and more preferably 3 to 6 carbon atoms, and an alkylene group having 6 to 18, preferably 6 to 14, and more preferably 6 to 10 carbon atoms,
   the (v) N-acyl amino acid esters comprise a branched alkyl group having 3 to 10, preferably 3 to 8, and more preferably 3 to 6 carbon atoms, and a linear or branched acyl group having 4 to 30, preferably 5 to 26, and more preferably 6 to 22 carbon atoms, and
   the (vi) alkyl lactates comprise a linear or branched alkyl group or a linear or branched alkenyl group, each having 4 to 30, preferably 5 to 26, and more preferably 6 to 22 carbon atoms,
and
the weight ratio of the amount of the (b) diol having at least one carbon chain comprising 3 or more carbon atoms, with the total number of carbon atoms of 5 or 6, to the amount of the (a) oil is 4 or more, preferably 5 or more, and more preferably 6 or more.

The composition according to the present invention is stable over time and can show a transparent or slightly translucent aspect.

The composition according to the present invention is stable over time, even under temperature changes, e.g., changes from hot to cool temperatures and vice versa, such that they do not cause any phase separation, while maintaining uniform appearance.

The composition according to the present invention comprises at least one oil. Accordingly, the composition according to the present invention can enhance glow of the skin when being applied onto the skin.

The composition according to the present invention can include a relatively large amount of oil. Therefore, the composition according to the present invention can provide better cosmetic effects derived from the relatively larger amount of oil, such as a further enhanced glow of the skin.

The composition according to the present invention can also provide fresh texture when being used. Thus, the composition according to the present invention can provide less greasy texture.

The composition according to the present invention can be transparent or slightly translucent, but preferably transparent.

The composition according to the present invention does not need to include any surfactant. Thus, the amount of surfactant(s) in the composition can be limited, such as 1% by weight or less, preferably 0.1% by weight or less, and more preferably 0.01% by weight or less, relative to the total weight of the composition. It is most preferable that the composition according to the present invention comprises no surfactant.

It has been well known that surfactants can provide greasy texture and stimulation to skin.

Thus, if the composition according to the present invention includes only a limited amount of surfactant(s), the composition according to the present invention can provide even less greasy texture.

In addition, if the composition according to the present invention includes only a limited amount of surfactant(s), the composition according to the present invention can provide less stimulation to skin.

Hereinafter, the composition according to the present invention will be explained in a more detailed manner.

### [Oil]

The composition according to the present invention may comprise (a) at least one oil. If two or more oils are used, they may be the same or different.

Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

According to the present invention, the (a) oil is selected from the group consisting of (i) fatty alcohols, (ii) fatty acids, (iii) monovalent carboxylic acid monoesters, (iv) divalent carboxylic acid diesters, (v) N-acyl amino acid esters, and (vi) alkyl lactates, under the following conditions:
the (i) fatty alcohols and the (ii) fatty acids comprise a branched alkyl group or a linear or branched alkenyl group, each having 4 to 30, preferably 5 to 26, and more preferably 6 to 22 carbon atoms,
the (iii) monovalent carboxylic acid monoesters comprise a branched alkyl group or a linear or branched alkenyl group, each having 4 to 30, preferably 5 to 26, and more preferably 6 to 22 carbon atoms,
the (iv) divalent carboxylic acid diesters comprise at least one, preferably two, branched alkyl group having 3 to 10, preferably 3 to 8, and more preferably 3 to 6 carbon atoms, and an alkylene group having 6 to 18, preferably 6 to 14, and more preferably 6 to 10 carbon atoms,
the (v) N-acyl amino acid esters comprise a branched alkyl group having 3 to 10, preferably 3 to 8, and more preferably 3 to 6 carbon atoms, and a linear or branched acyl group having 4 to 30, preferably 5 to 26, and more preferably 6 to 22 carbon atoms, and
the (vi) alkyl lactates comprise a linear or branched alkyl group or a linear or branched alkenyl group, each having 4 to 30, preferably 5 to 26, and more preferably 6 to 22 carbon atoms.

In the present specification, the “alkyl group” refers to a linear or branched monovalent saturated aliphatic hydrocarbon group.

As the linear alkyl group having 4 to 30 carbon atoms, mention may be made of, for example, an n-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, an n-nonadecyl group, and an n-eicosyl group.

As the branched alkyl group having 4 to 30 carbon atoms, mention may be made of, for example, an isobutyl group, a sec-butyl group, a tert-butyl group, an isopentyl group, a neopentyl group, an isohexyl group, an neohexyl group, an isoheptyl group, a neoheptyl group, an isooctyl group, an isononyl group, an isodecyl group, an isoundecyl group, an isododecyl group, an isotridecyl group, an isotetradecyl group, an isopentadecyl group, an isohexadecyl group, an isoheptadecyl group, an isooctadecyl group, an isononadecyl group, an isoeicosyl group.

As the branched alkyl group having 3 to 10 carbon atoms, mention may be made of, for example, an isopropyl group, an isobutyl group, a sec-butyl group, a tert-butyl group, an isopentyl group, a neopentyl group, an isohexyl group, an neohexyl group, an isoheptyl group, a neoheptyl group, an isooctyl group, an isononyl group, and an isodecyl group.

In the present specification, the “alkenyl group” refers to a linear or branched monovalent unsaturated aliphatic hydrocarbon group having at least one carbon-carbon double bond.

As the linear alkenyl group having 4 to 30 carbon atoms, mention may be made of, for example, a butenyl group, a pentenyl group, a hexenyl group, a heptenyl group, a heptadienyl group, a heptatrienyl group, an octenyl group, an octadienyl group, an octatrienyl group, a nonenyl group, a nonadienyl group, a nonatrienyl group, a decenyl group, a decadienyl group, a decatrienyl group, an undecenyl group, an undecadienyl group, an undecatrienyl group, a dodecenyl group, a dodecadienyl group, a dodecatrienyl group, a tridecenyl group, a tridecadienyl group, a tridecatrienyl group, a tetradecenyl group, a tetradecadienyl group, a tetradecadienyl group, a tetradecatrienyl group, a pentadecenyl group, a pentadecadienyl group, a pentadecatrienyl group, a hexadecenyl group, a hexadecadienyl group, a hexadecatrienyl group, a heptadecenyl group, a heptadecadienyl group, a heptadecatrienyl group, an octadecenyl group, an octadecadienyl group, an octadecatrienyl group, a nonadecenyl group, a nonadecadienyl group, a nonadecatrienyl group and an eicosenyl group.

As the branched alkenyl group having 4 to 30 carbon atoms, mention may be made of, for example, an isobutenyl group, a sec-butenyl group, an isopentenyl group, an isohexenyl group, an isoheptenyl group, an isooctenyl group, an isononenyl group, an isodecenyl group, an isodecadienyl group, an isodecatrienyl group, an isoundecenyl group, an isoundecadienyl group, an isoundecatrienyl group, an isododecenyl group, an isododecadienyl group, an isododecatrienyl group, an isotridecenyl group, an isotridecadienyl group, an isotridecatrienyl group, an isotetradecenyl group, an isotetradecadienyl group, an isotetradecadienyl group, an isotetradecatrienyl group, an isopentadecenyl group, an isopentadecadienyl group, an isopentadecatrienyl group, an isohexadecenyl group, an isohexadecadienyl group, an isohexadecatrienyl group, an isoheptadecenyl group, an isoheptadecadienyl group, an isoheptadecatrienyl group, an isooctadecenyl group, an isooctadecadienyl group, an isooctadecatrienyl group, an isononadecenyl group, an isononadecadienyl group, an isononadecatrienyl group and an isoeicosenyl group.

In the present specification, the “acyl group” refers to R—CO— group wherein R denotes a linear or branched monovalent saturated aliphatic hydrocarbon group (alkyl group).

As the linear or branched acyl group having 4 to 30 carbon atoms, mention may be made of, for example, a butanoyl group, an iso-butanoyl group, a sec-butanoyl group, a tert-butanoyl group, an n-pentanoyl group, an isopentanoyl group, an neopentanoyl group, an n-hexanoyl group, an isohexanoyl group, a neohexanoyl group, an n-heptanoyl group, an isoheptanoyl group, a neoheptanoyl group, an n-octanoyl group, an isooctanoyl group, an n-nonanoyl group, an isononanoyl group, an n-decanoyl group, an isodecanoyl group, an n-undecanoyl group, an isoundecanoyl group, an n-dodecanoyl group (lauroyl group), an isododecanoyl group, an n-tridecanoyl group, an isotridodecanoyl group, an n-tetradecanoyl group (myristoyl group), an isotetradecanoyl group, an n-pentadecanoyl group, an isopentadecanoyl group, an n-hexadecanoyl group (palmitoyl group), an isohexadecanoyl group, an n-heptadecanoyl group, an isohexadecanoyl group, an n-octadecanoyl group (stearoyl group), an isooctadecanoyl group, an n-nonadecanoyl group, an isononadecanoyl group, an n-eicosanoyl group, and an isoeicosanoyl group.

The (i) fatty alcohol may be represented by the following general formula (1):

R¹—OH (1)
wherein
R¹ represents a branched alkyl group, or a linear or branched alkenyl group, each having 4 to 30, preferably 5 to 26, and more preferably 6 to 22 carbon atoms.

As examples of the branched alkyl group, and the linear or branched alkenyl group, each having 4 to 30 carbon atoms, mention may be made of those listed above.

As the (i) fatty alcohol represented by the formula (1), mention may be made of, for example, 2-ethylhexanol, palmitoleyl alcohol, isostearyl alcohol, elaidyl alcohol, oleyl alcohol, linoleyl alcohol, ricinoleyl alcohol, erucyl alcohol, and a mixture thereof.

It is preferable that the (i) fatty alcohol be selected from the group consisting of isostearyl alcohol, oleyl alcohol, and a mixture thereof.

The (ii) fatty acid may be represented by the following general formula (2):

R¹—COOH (2)
wherein
R¹ represents a branched alkyl group, or a linear or branched alkenyl group, each having 4 to 30, preferably 5 to 26, and more preferably 6 to 22 carbon atoms.

As examples of the branched alkyl group, and the linear or branched alkenyl group, each having 4 to 30 carbon atoms, mention may be made of those listed above.

As the (ii) fatty acid represented by the formula (2), mention may be made of, for example, isocaproic acid, isononanoic acid, crotonic acid, isolauric acid, myristoleic acid, isopalmitic acid, palmitoleic acid, isostearic acid, elaidic acid, oleic acid, linoleic acid, linolenic acid, arachidonic acid, eicosapentaenoic acid, tetracosapentaenoic acid, docosahexaenoic acid, nervonic acid, and a mixture thereof.

It is preferable that the (ii) fatty acid be selected from the group consisting of isostearic acid, oleic acid, and a mixture thereof.

The (iii) monovalent carboxylic acid monoester may be represented by the following general formula (3):

R²—COO—R³ (3)
wherein
R² represents a branched alkyl group or a linear or branched alkenyl group, each having 4 to 30, preferably 5 to 26, and more preferably 6 to 22 carbon atoms, and
R³ represents a branched alkyl group having 3 to 10, preferably 3 to 8, and more preferably 3 to 6 carbon atoms.

As examples of the branched alkyl group and the linear or branched alkenyl group, each having 4 to 30 carbon atoms, mention may be made of those listed above.

As examples of the branched alkyl group having 3 to 10 carbon atoms, mention may be made of those listed above.

As the (iii) monovalent carboxylic acid monoester represented by the formula (3), mention may be made of, for example, isopropyl palmitate, isopropyl myristate, isocetyl stearate, 2-ethylhexyl isononanoate, isononyl isononanoate, isodecyl neopentanoate, isostearyl neopentanoate, and a mixture thereof.

It is preferable that the (iii) monovalent carboxylic acid monoester be isopropyl isostearate.

The (iv) divalent carboxylic acid diester may be represented by the following general formula (4):

R²—OC(═O)—R⁴—C(═O)O—R³ (4)
wherein
R² and R³ independently represent a branched alkyl group having 3 to 10, preferably 3 to 8, and more preferably 3 to 6 carbon atoms, and
R⁴ represents a linear or branched alkylene group having 6 to 18, preferably 6 to 14, and more preferably 6 to 10 carbon atoms.

As examples of the branched alkyl group having 3 to 10 carbon atoms, mention may be made of those listed above.

As examples of the linear or branched alkylene group having 2 to 10 carbon atoms, mention may be made of, for example, an ethylene group, a propylene group, a butylene group, a pentylene group, a hexylene group, a heptylene group, an octylene group, a nonylene group, and a decanylene group.

It is preferable that the (iv) divalent carboxylic acid diester be selected from the group consisting of diisopropyl adipate, diisopropyl sebacate, and a mixture thereof.

The (v) N-acyl amino acid ester may be represented by the following general formula (5):

R²—N(R⁵)—CH(R⁶)—(CH₂)ₙ—COO—R³ (5)
wherein
R² represents a linear or branched acyl group having 4 to 30, preferably 5 to 26, and more preferably 6 to 22 carbon atoms,
R³ represents a branched alkyl group having 3 to 10, preferably 3 to 8, and more preferably 3 to 6 carbon atoms,
R⁵ represents a hydrogen atom, or a linear or branched alkyl group having 1 to 4 carbon atoms,
R⁶ represents a hydrogen atom, or a linear or branched alkyl group having 1 to 4 carbon atoms which may be substituted with a hydroxy group or a phenyl group, and
n represents an integer from 0 to 2.

As examples of the linear or branched acyl group having 4 to 30 carbon atoms, mention may be made of those listed above.

As examples of the branched alkyl group having 3 to 10 carbon atoms, mention may be made of those listed above.

As examples of a linear or branched alkyl group having 1 to 4 carbon atoms, mention may be made of a methyl group, an ethyl group, a propyl group, an isopropyl group, a n-butyl group, an iso-butyl group, a sec-butyl group, and a tert-butyl group.

As the (v) N-acyl amino acid ester represented by the formula (5), mention may be made of, for example, N-capryloyl sarcosine isopropyl ester, N-caprynoyl sarcosine isopropyl ester, N-lauroyl sarcosine isopropyl ester, N-coconut oil fatty acid acyl sarcosine isopropyl ester (N-cocoyl sarcosine isopropyl ester), N-myristoyl sarcosine isopropyl ester, N-palmitoyl sarcosine isopropyl ester, N-stearoyl sarcosine isopropyl ester, N-capryloyl sarcosine isobutyl ester, N-caprynoyl sarcosine isobutyl ester, N-lauroyl sarcosine isobutyl ester, N-coconut oil fatty acid acyl sarcosine isobutyl ester (N-cocoyl sarcosine isobutyl ester), N-myristoyl sarcosine isobutyl ester, N-palmitoyl sarcosine isobutyl ester, N-stearoyl sarcosine isobutyl ester, N-capryloyl glycine isopropyl ester, N-caprynoyl glysine isopropyl ester, N-lauroyl glycine isopropyl ester, N-coconut oil fatty acid acyl glycine isopropyl ester (N-cocoyl glycine isopropyl ester), N-myristoyl glycine isopropyl ester, N-palmitoyl glycine isopropyl ester, N-stearoyl glycine isopropyl ester, N-capryloyl alanine isopropyl ester, N-caprinoyl alanine isopropyl ester, N-lauroyl alanine isopropyl ester, N-coconut oil fatty acid acyl alanine isopropyl ester (N-cocoyl alanine isopropyl ester), N-myristoyl alanine isopropyl ester, N-palmitoylalanine isopropyl ester, N-stearoylalanine isopropyl ester, N-lauroyl-β-alanine isopropyl ester, N-lauroyl-N-methyl-β-alanine isopropyl ester, N-coconut oil fatty acid acyl-N-methyl-β-alanine isopropyl ester (N-cocoyl-N-methyl-β-alanine isopropyl ester) and the like.

It is preferable that the (v) N-acyl amino acid ester be isopropyl lauroyl sarcosinate. As isopropyl lauroyl sarcosine, a commercial product such as Eldew® SL-205 by Ajinomoto may be used.

The (vi) alkyl lactate may be represented by the following general formula (6):

CH₃—C(OH)—COO—R⁷ (6)
wherein
R⁷ represents a linear or branched alkyl group or a linear or branched alkenyl group, each having 4 to 30, preferably 5 to 26, and more preferably 6 to 22 carbon atoms.

As examples of the linear or branched alkyl group or the linear or branched alkenyl group, each having 4 to 30 carbon atoms, mention may be made of those listed above.

As the examples of the (vi) alkyl lactate represented by the formula (6), mention may be made of, for example, capryl lactate, caprylyl lactate, lauryl lactate, myristyl lactate, palmityl lactate, stearyl lactate, isostearyl lactate, oleyl lactate, and linoleyl lactate.

It is preferable that the (vi) alkyl lactate be selected from the group consisting of lauryl lactate, isostearyl lactate, and a mixture thereof.

The amount of the (a) oil(s) in the composition according to the present invention may be 0.5% by weight or more, preferably 1% by weight or more, and more preferably 2% by weight or more, relative to the total weight of the composition.

On the other hand, the amount of the (a) oil(s) in the composition according to the present invention may be 25% by weight or less, preferably 20% by weight or less, and more preferably 15% by weight or less, relative to the total weight of the composition.

Thus, the amount of the (a) oil(s) in the composition according to the present invention may be from 0.5% to 25% by weight, preferably from 1% to 20% by weight, and more preferably from 2% to 15% by weight, relative to the total weight of the composition.

### [Diol]

The composition according to the present invention comprises at least one (b) diol(s) having at least one carbon chain comprising 3 or more carbon atoms, with the total number of carbon atoms of 5 or 6. Two or more (b) diol(s) having at least one carbon chain comprising 3 or more carbon atoms, with the total number of carbon atoms of 5 or 6, may be used.

The term “diol” here means a compound which has two alcohol functions. In other words, diol is an alcohol having two hydroxy groups.

The number of carbon atoms in the carbon chain may be 5 or 6.

The carbon chain comprising 3 or more carbon atoms in the ingredient (b) may be straight or branched. The carbon chain may not be interrupted by a heteroatom such as an oxygen atom, a sulfur atom and a nitrogen atom. Thus, the carbon chain may comprise consecutive carbon atoms.

It is preferable that the number of carbon chain comprising 3 or more carbon atoms in the ingredient (b) be 1 or 2.

The total number of carbon atoms in the ingredient (b) is 5 or 6.

The (b) diol(s) having at least one carbon chain comprising 3 or more carbon atoms, with the total number of carbon atoms of 5 or 6, may have a divalent straight or branched hydrocarbon group. The divalent straight or branched hydrocarbon group which may be present in the ingredient (b) may be saturated or unsaturated. It is preferable that the (b) diol(s) having at least one carbon chain comprising 3 or more carbon atoms, with the total number of carbon atoms of 5 or 6, comprise a divalent straight or branched saturated hydrocarbon group, and more preferably a straight or branched alkylene group, such as a propylene group, a pentylene group and a hexylene group.

The (b) diol(s) having at least one carbon chain comprising 3 or more carbon atoms, with the total number of carbon atoms of 5 or 6, may have a linear, branched or cyclic molecular structure, as long as it has the carbon chain comprising 3 or more carbon atoms, and the total number of carbon atoms is 5 or 6.

The (b) diol(s) having at least one carbon chain comprising 3 or more carbon atoms, with the total number of carbon atoms of 5 or 6, may be a C₅₋₆ diol, comprising 2 hydroxy groups.

It is preferable that the (b) diol(s) having at least one carbon chain comprising 3 or more carbon atoms, with the total number of carbon atoms of 5 or 6, be selected from the group consisting of dipropyleneglycol, pentyleneglycol, hexyleneglycol, and a mixture thereof.

It is more preferable that the (b) diol(s) having at least one carbon chain comprising 3 or more carbon atoms, with the total number of carbon atoms of 5 or 6, be selected from the group consisting of pentyleneglycol, hexyleneglycol, and a mixture thereof.

The amount of the (b) diol(s) having at least one carbon chain comprising 3 or more carbon atoms, with the total number of carbon atoms of 5, in the composition according to the present invention may be 40% by weight or more, preferably 50% by weight or more, more preferably 60% by weight or more, and even more preferably 70% by weight or more, relative to the total weight of the composition.

The amount of the (b) diol(s) having at least one carbon chain comprising 3 or more carbon atoms, with the total number of carbon atoms of 6, in the composition according to the present invention may be 60% by weight or more, and preferably 70% by weight or more, relative to the total weight of the composition.

On the other hand, the amount of the (b) diol(s) having at least one carbon chain comprising 3 or more carbon atoms, with the total number of carbon atoms of 5 or 6, in the composition according to the present invention may be 85% by weight or less, preferably 80% by weight or less, and more preferably 75% by weight or less, relative to the total weight of the composition.

### [Diol/Oil Weight Ratio]

The weight ratio of the amount of the (b) diol(s) having at least one carbon chain comprising 3 or more carbon atoms, with the total number of carbon atoms of 5 or 6 to the amount of the (a) oil(s) is 4 or more, preferably 5 or more, and more preferably 6 or more.

On the other hand, the weight ratio of the amount of the (b) diol(s) having at least one carbon chain comprising 3 or more carbon atoms, with the total number of carbon atoms of 5 or 6 to the amount of the (a) oil(s) may be 20 or less, preferably 15 or less, and more preferably 10 or less.

Thus, the weight ratio of the amount of the (b) diol(s) having at least one carbon chain comprising 3 or more carbon atoms, with the total number of carbon atoms of 5 or 6 to the amount of the (a) oil(s) may be from 4 to 20, preferably from 5 to 15, and more preferably from 6 to 10.

### [Water]

The composition according to the present invention comprises (c) water.

The amount of the (c) water in the composition according to the present invention may be 10% by weight or more, preferably 15% by weight or more, and more preferably 20% by weight or more, relative to the total weight of the composition.

On the other hand, the amount of the (c) water in the composition according to the present invention may be 60% by weight or less, preferably 55% by weight or less, and more preferably 50% by weight or less, relative to the total weight of the composition.

The amount of (c) water in the composition according to the present invention may range from 10% to 60% by weight, preferably from 15% to 55% by weight, more preferably from 20% to 50% by weight, relative to the total weight of the composition.

### [Monovalent Alcohol]

The composition according to the present invention may further comprise (d) at least one monovalent alcohol. Two or more such alcohols may be used in combination. Thus, a single type of such alcohol or a combination of different types of such alcohol may be used.

The (d) monovalent alcohol is preferably in the form of a liquid at ambient temperature such as 25° C. under atmospheric pressure (760 mmHg or 105 Pa).

The term “monovalent alcohol” here means an alcohol having one hydroxy group.

The (d) monovalent alcohol may be non-aromatic (aliphatic) or aromatic.

The non-aromatic monovalent alcohol is preferably a saturated or unsaturated, linear or branched lower aliphatic monovalent alcohol, more preferably a C₂-C₆ aliphatic monovalent alcohol, even more preferably a saturated or unsaturated, linear or branched C₂-C₅ aliphatic monovalent alcohol, and most preferably a saturated or unsaturated, linear or branched C₂-C₄ aliphatic monovalent alcohol. Preferred non-aromatic monovalent alcohols are ethanol, isopropanol and mixtures thereof.

The aromatic monovalent alcohol is preferably selected from the group consisting of benzyl alcohol, phenethylalcohol, diphenyl ethanol, cinnamyl alcohol, tryptophol, 3-nitrobenzylalcohol, veratryl alcohol, benzoin and mixtures thereof.

It is preferable that the (d) monovalent alcohol not be a fatty alcohol or higher alcohol.

It is preferable that the (d) monovalent alcohol be a C₂₋₄ aliphatic monovalent alcohol. It is more preferable that the (d) monovalent alcohol be ethanol.

The amount of the (d) monovalent alcohol(s) in the composition according to the present invention may be 1% by weight or more, preferably 5% by weight or more, and more preferably 10% by weight or more, relative to the total weight of the composition.

On the other hand, the amount of the (d) monovalent alcohol(s) in the composition according to the present invention may be 60% by weight or less, preferably 55% by weight or less, and more preferably 50% by weight or less, relative to the total weight of the composition.

The amount of the (d) monovalent alcohol(s) in the composition according to the present invention may range from 1% to 60% by weight, preferably from 5% to 55% by weight, and more preferably from 10% to 50% by weight, relative to the total weight of the composition.

The weight ratio of the total amount of the (b) diol(s) having at least one carbon chain comprising 3 or more carbon atoms, with the total number of carbon atoms of 5 or 6 and the (d) monovalent alcohol(s) to the amount of the (a) oil(s) may be 4 or more, preferably 5 or more, and more preferably from 6 or more.

The weight ratio of the total amount of the (b) diol(s) having at least one carbon chain comprising 3 or more carbon atoms, with the total number of carbon atoms of 5 or 6 and the (d) monovalent alcohol(s) to the amount of the (a) oil(s) may be 20 or less, preferably 15 or less, and more preferably 10 or less.

The weight ratio of the total amount of the (b) diol(s) having at least one carbon chain comprising 3 or more carbon atoms, with the total number of carbon atoms of 5 or 6 and the (d) monovalent alcohol(s) to the amount of the (a) oil(s) may range from 4 to 20, preferably from 5 to 15, and more preferably from 6 to 10.

### [Optional Ingredients]

The composition according to the present invention may further comprise at least one optional ingredient usually used in the field of cosmetics, chosen, for example, film-forming polymers, solvents, dyes, pigments, UV filters, antioxidants, preserving agents, pH adjusting agents, and mixtures thereof.

The composition according to the present invention may include one or several cosmetically acceptable organic solvents, which may be diols other than the ingredient (b), such as ethylene glycol; other polyols such as glycerol, sugar, and sugar alcohols; and ethers such as ethylene glycol monomethyl, monoethyl, and monobutyl ethers, propylene glycol monomethyl, monoethyl, and monobutyl ether, and butylene glycol monomethyl, monoethyl, and monobutyl ethers.

The amount of the organic solvent(s) in the composition according to the present invention may be 0.01% by weight or more, preferably 0.1% by weight or more, and more preferably 1% by weight or more, relative to the total weight of the composition.

On the other hand, the amount of the organic solvent(s) in the composition according to the present invention may be 30% by weight or less, preferably 20% by weight or less, and more preferably 10% by weight or less, relative to the total weight of the composition.

Thus, the amount of the organic solvent(s) in the composition according to the present invention may range from 0.01% to 30% by weight, preferably from 0.1% to 20% by weight, and more preferably from 1% to 10% by weight, relative to the total weight of the composition.

### (Preparation)

The composition according to the present invention can be prepared by mixing the essential ingredient(s) as explained above, and optional ingredient(s), if necessary, as explained above.

The method and means to mix the above essential and optional ingredients are not limited. Any conventional method and means can be used to mix the above essential and optional ingredients to prepare the composition according to the present invention.

### [Form]

The form of the composition according to the present invention is not limited.

The composition according to the present invention can be thermodynamically stable, and may be considered as a single phase composition.

The composition according to the present invention can be transparent.

The transparency may be measured by measuring the turbidity (for example, turbidity can be measured with a 2100Q (marketed by Hach Company) having a round cell (25 mm in diameter and 60 mm height) and a tungsten filament lamp which can emit visible light (between 400 and 800 nm, preferably from 400 to 500 nm). The measurement can be performed on the undiluted composition. The blank may be determined with distilled water.

The composition according to the present invention has a turbidity of 120 NTU or less, preferably 90 NTU or less and more preferably 60 NTU or less.

### [Process and Use]

It is preferable that the composition according to the present invention be a cosmetic or dermatological composition, preferably a skin cosmetic composition, and more preferably a skin care cosmetic composition.

The composition according to the present invention can be used for a non-therapeutic process, such as a cosmetic process, for treating a skin, by being applied to the skin.

Thus, the present invention also relates to a cosmetic process for treating a keratin substance such as skin, comprising the step of applying the composition according to the present invention to the keratin substance.

The present invention may also relate to a use of the composition according to the present invention as a cosmetic product or in a cosmetic product such as skin care products.

In other words, the composition according to the present invention can be used, as it is, as a cosmetic product. Alternatively, the composition according to the present invention can be used as an element of a cosmetic product. For example the composition according to the present invention can be added to or combined with any other elements to form a cosmetic product.

The skin care product may be a lotion, a cream, a serum, and the like.

Another aspect of the present invention also relates to a process for preparing a composition, comprising a step of mixing:
(a) at least one oil;
(b) at least one diol having at least one carbon chain comprising 3 or more carbon atoms, with the total number of carbon atoms of 5 or 6; and
(c) water,
wherein
the (a) oil is selected from the group consisting of (i) fatty alcohols, (ii) fatty acids, (iii) monovalent carboxylic acid monoesters, (iv) divalent carboxylic acid diesters, (v) N-acyl amino acid esters, and (vi) alkyl lactates, provided that
   the (i) fatty alcohols and the (ii) fatty acids comprise a branched alkyl group or a linear or branched alkenyl group, each having 4 to 30, preferably 5 to 26, and more preferably 6 to 22 carbon atoms,
   the (iii) monovalent carboxylic acid monoesters comprise a branched alkyl group or a linear or branched alkenyl group, each having 4 to 30, preferably 5 to 26, and more preferably 6 to 22 carbon atoms,
   the (iv) divalent carboxylic acid diesters comprise at least one branched alkyl group having 3 to 10, preferably 3 to 8, and more preferably 3 to 6 carbon atoms, and an alkylene group having 6 to 18, preferably 6 to 14, and more preferably 6 to 10 carbon atoms,
   the (v) N-acyl amino acid esters comprise a branched alkyl group having 3 to 10, preferably 3 to 8, and more preferably 3 to 6 carbon atoms, and a linear or branched acyl group having 4 to 30, preferably 5 to 26, and more preferably 6 to 22 carbon atoms, and
   the (vi) alkyl lactates comprise a linear or branched alkyl group or a linear or branched alkenyl group, each having 4 to 30, preferably 5 to 26, and more preferably 6 to 22 carbon atoms,
and
the weight ratio of the amount of the (b) diol having at least one carbon chain comprising 3 or more carbon atoms, with the total number of carbon atoms of 5 or 6, to the amount of the (a) oil is 4 or more, preferably 5 or more, and more preferably 6 or more.

It is preferable that the mixing step be performed by a so-called low energy process without a special mechanical stirrer, such as a homogenizer which uses a large amount of energy. The low energy process can be performed by simply gently stirring the ingredients (a) to (c).

Another aspect of the present invention relates to a use of:
(a) at least one oil; and
(b) at least one diol having at least one carbon chain comprising 3 or more carbon atoms, with the total number of carbon atoms of 5 or 6,
in a composition, comprising
(c) water,
wherein
the (a) oil is selected from the group consisting of (i) fatty alcohols, (ii) fatty acids, (iii) monovalent carboxylic acid monoesters, (iv) divalent carboxylic acid diesters, (v) N-acyl amino acid esters, and (vi) alkyl lactates, provided that
   the (i) fatty alcohols and the (ii) fatty acids comprise a branched alkyl group or a linear or branched alkenyl group, each having 4 to 30, preferably 5 to 26, and more preferably 6 to 22 carbon atoms, and
   the (iii) monovalent carboxylic acid monoesters comprise a branched alkyl group or a linear or branched alkenyl group, each having 4 to 30, preferably 5 to 26, and more preferably 6 to 22 carbon atoms,
   the (iv) divalent carboxylic acid diesters comprise at least one branched alkyl group having 3 to 10, preferably 3 to 8, and more preferably 3 to 6 carbon atoms, and an alkylene group having 6 to 18, preferably 6 to 14, and more preferably 6 to 10 carbon atoms,
   the (v) N-acyl amino acid esters comprise a branched alkyl group having 3 to 10, preferably 3 to 8, and more preferably 3 to 6 carbon atoms, and a linear or branched acyl group having 4 to 30, preferably 5 to 26, and more preferably 6 to 22 carbon atoms and
   the (vi) alkyl lactates comprise a linear or branched alkyl group or a linear or branched alkenyl group, each having 4 to 30, preferably 5 to 26, and more preferably 6 to 22 carbon atoms,
and
the weight ratio of the amount of the (b) diol having at least one carbon chain comprising 3 or more carbon atoms, with the total number of carbon atoms of 5 or 6, to the amount of the (a) oil is 4 or more, preferably 5 or more, and more preferably 6 or more.
in order to render the composition stable and transparent or slightly translucent.

The above explanations regarding the ingredients (a) to (c), as well as the optional ingredients, for the composition according to the present invention can apply to those for the uses and processes according to the present invention. The explanations regarding the preparation and forms of the composition according to the present invention can also apply to those of the composition recited in the above uses and processes.

### EXAMPLES

The present invention will be described in more detail by way of examples which, however, should not be construed as limiting the scope of the present invention.

### Examples 1-32 and Comparative Examples 1-46

The following compositions according to Examples 1-32 and Comparative Examples 1-46 shown in Tables 1-9, were prepared by mixing the ingredients shown in Tables 1-9 as follows. The numerical values for the amounts of the ingredients shown in Tables 1-9 are all based on “% by weight” as active raw materials.

The weight ratio of the amount (weight) of the diol(s) having at least one carbon chain comprising 3 or more carbon atoms, with the total number of carbon atoms of 5 or 6, to the amount (weight) of the oil(s) in each composition (Diol(s)/Oil(s)) is shown in the line of “Ratio of Diol to Oil” in Tables 1~4 and 6-9.

The weight ratio of the total amount (weight) of the diol(s) having at least one carbon chain comprising 3 or more carbon atoms, with the total number of carbon atoms of 5 or 6 and the monovalent alcohol(s) to the amount (weight) of the oil ingredient(s) in each composition ((Diol(s)+Alcohol(s))/Oil(s)) is shown in the line of “Ratio of Diol+Alcohol to Oil” in Table 5.

TABLE 1

| | Comp. | Comp. | | | | | Comp. | Comp. | | |
|---|---|---|---|---|---|---|---|---|---|---|
| Ingredients | Ex. 1 | Ex. 2 | Ex. 1 | Ex. 2 | Ex. 3 | Ex. 4 | Ex. 3 | Ex. 4 | Ex. 5 | Ex. 6 |
| Pentylene Glycol (C5) | 20 | 30 | 40 | 50 | 60 | 70 | — | — | — | — |
| Hexylene Glycol (C6) | — | — | — | — | — | — | 20 | 30 | 60 | 70 |
| Diisopropyl Adipate | 10 | 10 | 10 | 10 | 10 | 10 | 10 | 10 | 10 | 10 |
| Water | qsp | qsp | qsp | qsp | qsp | qsp | qsp | qsp | qsp | qsp |
| | 100 | 100 | 100 | 100 | 100 | 100 | 100 | 100 | 100 | 100 |
| Ratio of Diol to Oil | 2 | 3 | 4 | 5 | 6 | 7 | 2 | 3 | 6 | 7 |
| Aspect | Poor | Poor | Good | Good | Good | Good | Poor | Poor | Good | Good |
| Turbidity | 1000 | 1000 | 23 | 44 | 15 | 1.2 | 1000 | 1000 | 13 | 19 |
| Stability | Poor | Poor | Good | Good | Good | Good | Poor | Poor | Good | Good |

TABLE 2

| | Comp. | Comp. | Comp. | Comp. | Comp. | Comp. |
|---|---|---|---|---|---|---|
| Ingredients | Ex. 5 | Ex. 6 | Ex. 7 | Ex. 8 | Ex. 9 | Ex. 10 |
| Ethylene Glycol (C2) | 20 | 30 | 40 | 50 | 60 | 70 |
| Diisopropyl Adipate | 10 | 10 | 10 | 10 | 10 | 10 |
| Water | qsp | qsp | qsp | qsp | qsp | qsp |
| | 100 | 100 | 100 | 100 | 100 | 100 |
| Ratio of Diol to Oil | 2 | 3 | 4 | 5 | 6 | 7 |
| Aspect | Poor | Poor | Poor | Poor | Poor | Poor |
| Turbidity | 1000 | 1000 | 1000 | 1000 | 1000 | 1000 |
| Stability | Poor | Poor | Poor | Poor | Poor | Poor |

TABLE 3

| | Comp. | Comp. | Comp. | Comp. | Comp. | Comp. | Comp. | Comp. | Comp. | Comp. | Comp. | Comp. |
|---|---|---|---|---|---|---|---|---|---|---|---|---|
| Ingredients | Ex. 11 | Ex. 12 | Ex. 13 | Ex. 14 | Ex. 15 | Ex. 16 | Ex. 17 | Ex. 18 | Ex. 19 | Ex. 20 | Ex. 21 | Ex. 22 |
| Butylene Glycol (C4) | 20 | 30 | 40 | 50 | 60 | 70 | — | — | — | — | — | — |
| Caprylyl Glycol (C8) | — | — | — | — | — | — | 20 | 30 | 40 | 50 | 60 | 70 |
| Diisopropyl Adipate | 10 | 10 | 10 | 10 | 10 | 10 | 10 | 10 | 10 | 10 | 10 | 10 |
| Water | qsp | qsp | qsp | qsp | qsp | qsp | qsp | qsp | qsp | qsp | qsp | qsp |
| | 100 | 100 | 100 | 100 | 100 | 100 | 100 | 100 | 100 | 100 | 100 | 100 |
| Ratio of Diol to Oil | 2 | 3 | 4 | 5 | 6 | 7 | 2 | 3 | 4 | 5 | 6 | 7 |
| Aspect | Poor | Poor | Poor | Poor | Poor | Poor | Poor | Poor | Poor | Poor | Poor | Poor |
| Turbidity | 1000 | 1000 | 1000 | 1000 | 1000 | 1000 | 1000 | 1000 | 1000 | 1000 | 1000 | 1000 |
| Stability | Poor | Poor | Poor | Poor | Poor | Poor | Poor | Poor | Poor | Poor | Poor | Poor |

TABLE 4

| | Comp. | Comp. | | | |
|---|---|---|---|---|---|
| Ingredients | Ex. 23 | Ex. 24 | Ex. 7 | Ex. 8 | Ex. 9 |
| Pentylene Glycol (C5) | 10 | 15 | 25 | 30 | 35 |
| Hexylene Glycol (C6) | 10 | 15 | 25 | 30 | 35 |
| Diisopropyl Adipate | 10 | 10 | 10 | 10 | 10 |
| Water | qsp | qsp | qsp | qsp | qsp |
| | 100 | 100 | 100 | 100 | 100 |
| Ratio of Diol to Oil | 2 | 3 | 5 | 6 | 7 |
| Aspect | Poor | Poor | Good | Good | Good |
| Turbidity | 1000 | 1000 | 26 | 12 | 2.6 |
| Stability | Poor | Poor | Good | Good | Good |

TABLE 5

| | Comp. | Comp. | | | |
|---|---|---|---|---|---|
| Ingredients | Ex. 25 | Ex. 26 | Ex. 10 | Ex. 11 | Ex. 12 |
| Pentylene Glycol (C5) | 10 | 15 | 25 | 30 | 35 |
| Ethanol | 10 | 15 | 25 | 30 | 35 |
| Diisopropyl Adipate | 10 | 10 | 10 | 10 | 10 |
| Water | qsp | qsp | qsp | qsp | qsp |
| | 100 | 100 | 100 | 100 | 100 |
| Ratio of Diol + | 2 | 3 | 5 | 6 | 7 |
| Alcohol to Oil | | | | | |
| Aspect | Poor | Poor | Good | Good | Good |
| Turbidity | 1000 | 1000 | 49 | 38 | 23 |
| Stability | Poor | Poor | Good | Good | Good |

TABLE 6

| | | | | | Comp. | Comp. | Comp. | Comp. | Comp. | Comp. | Comp. | Comp. | Comp. | Comp. |
|---|---|---|---|---|---|---|---|---|---|---|---|---|---|---|
| | Ex. | Ex. | Ex. | Ex. | Ex. | Ex. | Ex. | Ex. | Ex. | Ex. | Ex. | Ex. | Ex. | Ex. |
| Ingredients | 13 | 14 | 15 | 16 | 27 | 28 | 29 | 30 | 31 | 32 | 33 | 34 | 35 | 36 |
| Pentylene Glycol | 60 | 60 | — | — | 60 | 60 | 60 | 60 | 60 | — | — | — | — | — |
| Hexylene Glycol | — | — | 60 | 60 | — | — | — | — | — | 60 | 60 | 60 | 60 | 60 |
| Isostearyl Alcohol | 10 | — | 10 | — | — | — | — | — | — | — | — | — | — | — |
| (C18, branched) | | | | | | | | | | | | | | |
| Oleyl Alcohol | — | 10 | — | 10 | — | — | — | — | — | — | — | — | — | — |
| (C18, unsaturated) | | | | | | | | | | | | | | |
| Myristyl Alcohol | — | — | — | — | 10 | — | — | — | — | 10 | — | — | — | — |
| Cetyl Alcohol | — | — | — | — | — | 10 | — | — | — | — | 10 | — | — | — |
| Stearyl Alcohol | — | — | — | — | — | — | 10 | — | — | — | — | 10 | — | — |
| Arachidyl Alcohol | — | — | — | — | — | — | — | 10 | — | — | — | — | 10 | — |
| Behenyl Alcohol | — | — | — | — | — | — | — | — | 10 | — | — | — | — | 10 |
| Water | qsp | qsp | qsp | qsp | qsp | qsp | qsp | qsp | qsp | qsp | qsp | qsp | qsp | qsp |
| | 100 | 100 | 100 | 100 | 100 | 100 | 100 | 100 | 100 | 100 | 100 | 100 | 100 | 100 |
| Ratio of Diol to Oil | 6 | 6 | 6 | 6 | 6 | 6 | 6 | 6 | 6 | 6 | 6 | 6 | 6 | 6 |
| Aspect | Good | Good | Good | Good | Poor | Poor | Poor | Poor | Poor | Poor | Poor | Poor | Poor | Poor |
| Turbidity | 13 | 18 | 29 | 48 | 1000 | 1000 | 1000 | 1000 | 1000 | 1000 | 1000 | 1000 | 1000 | 1000 |
| Stability | Good | Good | Good | Good | Poor | Poor | Poor | Poor | Poor | Poor | Poor | Poor | Poor | Poor |

TABLE 7

| | | | | | Comp. | Comp. | Comp. | Comp. | Comp. | Comp. | Comp. | Comp. | Comp. | Comp. |
|---|---|---|---|---|---|---|---|---|---|---|---|---|---|---|
| | Ex. | Ex. | Ex. | Ex. | Ex. | Ex. | Ex. | Ex. | Ex. | Ex. | Ex. | Ex. | Ex. | Ex. |
| Ingredients | 17 | 18 | 19 | 20 | 37 | 38 | 39 | 40 | 41 | 42 | 43 | 44 | 45 | 46 |
| Pentylene Glycol | 60 | 60 | — | — | 60 | 60 | 60 | 60 | 60 | — | — | — | — | — |
| Hexylene Glycol | — | — | 60 | 60 | — | — | — | — | — | 60 | 60 | 60 | 60 | 60 |
| Isostearic Acid | 10 | — | 10 | — | — | — | — | — | — | — | — | — | — | — |
| (C18, branched) | | | | | | | | | | | | | | |
| Oleic Acid | — | 10 | — | 10 | — | — | — | — | — | — | — | — | — | — |
| (C18, unsaturated) | | | | | | | | | | | | | | |
| Myristic Acid | — | — | — | — | 10 | — | — | — | — | 10 | — | — | — | — |
| Palmitic Acid | — | — | — | — | — | 10 | — | — | — | — | 10 | — | — | — |
| Stearic Acid | — | — | — | — | — | — | 10 | — | — | — | — | 10 | — | — |
| Arachidic Acid | — | — | — | — | — | — | — | 10 | — | — | — | — | 10 | — |
| Behenic Acid | — | — | — | — | — | — | — | — | 10 | — | — | — | — | 10 |
| Water | qsp | qsp | qsp | qsp | qsp | qsp | qsp | qsp | qsp | qsp | qsp | qsp | qsp | qsp |
| | 100 | 100 | 100 | 100 | 100 | 100 | 100 | 100 | 100 | 100 | 100 | 100 | 100 | 100 |
| Ratio of Diol to Oil | 6 | 6 | 6 | 6 | 6 | 6 | 6 | 6 | 6 | 6 | 6 | 6 | 6 | 6 |
| Aspect | Good | Good | Good | Good | Poor | Poor | Poor | Poor | Poor | Poor | Poor | Poor | Poor | Poor |
| Turbidity | 23 | 39 | 18 | 36 | 1000 | 1000 | 1000 | 1000 | 1000 | 1000 | 1000 | 1000 | 1000 | 1000 |
| Stability | Good | Good | Good | Good | Poor | Poor | Poor | Poor | Poor | Poor | Poor | Poor | Poor | Poor |

TABLE 8

| Ingredients | Ex. 21 | Ex. 22 | Ex. 23 | Ex. 24 | Ex. 25 | Ex. 26 | Ex. 27 | Ex. 28 |
|---|---|---|---|---|---|---|---|---|
| Pentylene Glycol | 60 | — | 60 | — | 60 | — | 60 | — |
| Hexylene Glycol | — | 60 | — | 60 | — | 60 | — | 60 |
| Isopropyl Isostearate | — | — | — | — | — | — | 10 | 10 |
| (C18, branched, | | | | | | | | |
| monoester) | | | | | | | | |
| Diisopropyl Adipate | 10 | 10 | — | — | — | — | — | — |
| (C6, diester) | | | | | | | | |
| Diisopropyl | — | — | 10 | 10 | — | — | — | — |
| Sebacate | | | | | | | | |
| (C10, diester) | | | | | | | | |
| Isopropyl Lauroyl | — | — | — | — | 10 | 10 | — | — |
| Sarcosinate | | | | | | | | |
| (C12) | | | | | | | | |
| Water | qsp | qsp | qsp | qsp | qsp | qsp | qsp | qsp |
| | 100 | 100 | 100 | 100 | 100 | 100 | 100 | 100 |
| Ratio of Diol to Oil | 6 | 6 | 6 | 6 | 6 | 6 | 6 | 6 |
| Aspect | Good | Good | Good | Good | Good | Good | Good | Good |
| Turbidity | 29 | 23 | 15 | 12 | 36 | 23 | 8.9 | 2.5 |
| Stability | Good | Good | Good | Good | Good | Good | Good | Good |

TABLE 9

| Ingredients | Ex. 29 | Ex. 30 | Ex. 31 | Ex. 32 |
|---|---|---|---|---|
| Pentylene Glycol | 60 | — | 60 | — |
| Hexylene Glycol | — | 60 | — | 60 |
| Lauryl Lactate | 10 | 10 | — | — |
| (C12, lactate) | | | | |
| Isostearyl Lactate | — | — | 10 | 10 |
| (C18, branched, lactate) | | | | |
| Water | qsp | qsp | qsp | qsp |
| | 100 | 100 | 100 | 100 |
| Ratio of Diol to Oil | 6 | 6 | 6 | 6 |
| Aspect | Good | Good | Good | Good |
| Turbidity | 14 | 12 | 11 | 15 |
| Stability | Good | Good | Good | Good |

### [Evaluations]

### (Aspect)

The aspect of each of the compositions according to Examples 1-32 and Comparative Examples 1-46 was evaluated by visual observation of the aspect of the composition which was kept at 55° C. for 2 weeks and at 4° C. for 2 weeks, based on the following criteria.
Good: Uniform
Poor: Non-Uniform (phase separation was recognized)

The results are shown in Tables 1-9.

### (Turbidity)

The turbidity of each of the compositions according to Examples 1-32 and Comparative Examples 1-46, which was kept at 55° C. for 2 weeks and at 4° C. for 2 weeks, was evaluated by a 2100Q (Hach Company) at room temperature (20-25° C.).

The results are shown in Tables 1-9.

### (Stability)

The stability of each of the compositions according to Examples 1-32 and Comparative Examples 1-46 was evaluated by visual observation of the aspect of the composition which was kept at 55° C. for 2 weeks and at 4° C. for 2 weeks, based on the following criteria.
Good: Transparent
Poor: Not Transparent

The results of the stability evaluation are shown in Tables 1-9.

### SUMMARY

Table 1 demonstrates that
the weight ratio of the amount of the (b) diol having at least one carbon chain comprising 3 or more carbon atoms, with the total number of carbon atoms of 5 or 6, to the amount of the (a) oil should be 4 or more, preferably 5 or more, and more preferably 6 or more.

Tables 2 and 3 demonstrate that the (b) diol should have at least one carbon chain comprising 3 or more carbon atoms, with the total number of carbon atoms of 5 or 6.

Table 4 demonstrates that a mixture of the (b) diols can provide the desired effects of the present invention, if the weight ratio of the amount of the (b) diols to the amount of the (a) oil is 4 or more.

Table 5 demonstrates that the composition including the (d) monovalent alcohol can provide the desired effects of the present invention if the weight ratio of the total amount of the (b) diol(s) and the (d) monovalent alcohol to the amount of the (a) oil is 4 or more.

Table 6 demonstrates that (i) fatty alcohols with a branched alkyl group or a linear or branched alkenyl group, each having 4 to 30, preferably 5 to 26, and more preferably 6 to 22 carbon atoms should be used.

Table 7 demonstrates that (ii) fatty acids with a branched alkyl group or a linear or branched alkenyl group, each having 4 to 30, preferably 5 to 26, and more preferably 6 to 22 carbon atoms should be used.

Table 8 demonstrates that (iii) monovalent carboxylic acid monoesters with a branched alkyl group or a linear or branched alkenyl group, each having 4 to 30, preferably 5 to 26, and more preferably 6 to 22 carbon atoms, (iv) divalent carboxylic acid diesters with at least one branched alkyl group having 3 to 10, preferably 3 to 8, and more preferably 3 to 6 carbon atoms, and (v) N-acyl amino acid esters, with a branched alkyl group having 3 to 10, preferably 3 to 8, and more preferably 3 to 6 carbon atoms and a linear or branched acyl group having 4 to 30, preferably 5 to 26, and more preferably 6 to 22 carbon atoms, should be used.

Table 9 demonstrates that (iv) alkyl lactates with a linear or branched alkyl group or a linear or branched alkenyl group, each having 4 to 30, preferably 5 to 26, and more preferably 6 to 22 carbon atoms, should be used.

Thus, Tables 1-9 demonstrate that the use of
(a) at least one oil; and
(b) at least one diol having at least one carbon chain comprising 3 or more carbon atoms, with the total number of carbon atoms of 5 or 6,
in a composition, comprising
(c) water,
wherein
the (a) oil is selected from the group consisting of (i) fatty alcohols, (ii) fatty acids, (iii) monovalent carboxylic acid monoesters, (iv) divalent carboxylic acid diesters, (v) N-acyl amino acid esters, and (vi) alkyl lactates, provided that
   the (i) fatty alcohols and the (ii) fatty acids comprise a branched alkyl group or a linear or branched alkenyl group, each having 4 to 30, preferably 5 to 26, and more preferably 6 to 22 carbon atoms,
   the (iii) monovalent carboxylic acid monoesters comprise a branched alkyl group or a linear or branched alkenyl group, each having 4 to 30, preferably 5 to 26, and more preferably 6 to 22 carbon atoms,
   the (iv) divalent carboxylic acid diesters comprise at least one branched alkyl group having 3 to 10, preferably 3 to 8, and more preferably 3 to 6 carbon atoms, and an alkylene group having 6 to 18, preferably 6 to 14, and more preferably 6 to 10 carbon atoms,
   the (v) N-acyl amino acid esters comprise a branched alkyl group having 3 to 10, preferably 3 to 8, and more preferably 3 to 6 carbon atoms, and a linear or branched acyl group having 4 to 30, preferably 5 to 26, and more preferably 6 to 22 carbon atoms, and
   the (vi) alkyl lactates comprise a linear or branched alkyl group or a linear or branched alkenyl group, each having 4 to 30, preferably 5 to 26, and more preferably 6 to 22 carbon atoms,
and
the weight ratio of the amount of the (b) diol having at least one carbon chain comprising 3 or more carbon atoms, with the total number of carbon atoms of 5 or 6, to the amount of the (a) oil is 4 or more, preferably 5 or more, and more preferably 6 or more
can render the composition stable and transparent.

1. A composition, comprising:
(a) at least one oil;
(b) at least one diol having at least one carbon chain comprising 3 or more carbon atoms, with the total number of carbon atoms of 5 or 6; and
(c) water,
wherein
the (a) oil is selected from the group consisting of (i) fatty alcohols, (ii) fatty acids, (iii) monovalent carboxylic acid monoesters, (iv) divalent carboxylic acid diesters, (v) N-acyl amino acid esters, and (vi) alkyl lactates provided that
the (i) fatty alcohols and the (ii) fatty acids comprise a branched alkyl group or a linear or branched alkenyl group, each having 4 to 30 carbon atoms,
the (iii) monovalent carboxylic acid monoesters comprise a branched alkyl group or a linear or branched alkenyl group, each having 4 to 30 carbon atoms,
the (iv) divalent carboxylic acid diesters comprise at least one branched alkyl group having 3 to 10 carbon atoms, and an alkylene group having 6 to 18 carbon atoms,
the (v) N-acyl amino acid esters comprise a branched alkyl group having 3 to 10 carbon atoms, and a linear or branched acyl group having 4 to 30 carbon atoms, and
the (vi) alkyl lactates comprise a linear or branched alkyl group or a linear or branched alkenyl group, each having 4 to 30 carbon atoms,
and
the weight ratio of the amount of the (b) diol having at least one carbon chain comprising 3 or more carbon atoms, with the total number of carbon atoms of 5 or 6, to the amount of the (a) oil is 4 or more.

2. The composition according to claim 1, wherein the (i) fatty alcohol is represented by the following general formula (1):
R¹—OH (1)
wherein
R¹ represents a branched alkyl group, or a linear or branched alkenyl group, each having 4 to 30 carbon atoms.

3. The composition according to claim 1, wherein the (ii) fatty acid is represented by the following general formula (2):
R¹—COOH (2)
wherein
R¹ represents a branched alkyl group, or a linear or branched alkenyl group, each having 4 to 30 carbon atoms.

4. The composition according to claim 1, wherein the (iii) monovalent carboxylic acid monoester is represented by the following general formula (3):
R²—COO—R³ (3)
wherein
R² represents a branched alkyl group or a linear or branched alkenyl group, each having 4 to 30 carbon atoms, and
R³ represents a branched alkyl group having 3 to 10 carbon atoms.

5. The composition according to claim 1, wherein the (iv) divalent carboxylic acid diester is represented by the following general formula (4):
R²—OC(═O)—R⁴—C(═O)O—R³ (4)
wherein
R² and R³ independently represent a branched alkyl group having 3 to 10 carbon atoms, and
R⁴ represents a linear or branched alkylene group having 6 to 18 carbon atoms.

6. The composition according to claim 1, wherein the (v) N-acyl amino acid ester is represented by the following general formula (5):
R²—N(R⁵)—CH(R⁶)—(CH₂)ₙ—COO—R³ (5)
wherein
R² represents a linear or branched acyl group having 4 to 30 carbon atoms,
R³ represents a branched alkyl group having 3 to 10 carbon atoms,
R⁵ represents a hydrogen atom, or a linear or branched alkyl group having 1 to 4 carbon atoms,
R⁶ represents a hydrogen atom, or a linear or branched alkyl group having 1 to 4 carbon atoms which may be substituted with a hydroxy group or a phenyl group, and
n represents an integer from 0 to 2.

7. The composition according to claim 1, wherein the (vi) alkyl lactate is represented by the following general formula (6):
CH₃—C(OH)—COO—R⁷ (6)
wherein
R⁷ represents a linear or branched alkyl group or a linear or branched alkenyl group, each having 4 to 30 carbon atoms.

8. The composition according to claim 1, wherein the amount of the (a) oil(s) in the composition ranges from 0.5% to 25% by weight relative to the total weight of the composition.

9. The composition according to claim 1, wherein the (b) diol having at least one carbon chain comprising 3 or more carbon atoms, with the total number of carbon atoms of 5 or 6 is selected from the group consisting of dipropylene glycol, pentylene glycol, hexylene glycol, and a mixture thereof.

10. The composition according to claim 1, wherein the composition further comprises (d) at least one monovalent alcohol.

11. The composition according to claim 10, wherein the weight ratio of the total amount of the (b) diol(s) having at least one carbon chain comprising 3 or more carbon atoms, with the total number of carbon atoms of 5 or 6 and the (d) monovalent alcohol(s) to the amount of the (a) oil is 4 or more.

12. The composition according to claim 1, wherein the composition has a turbidity of 120 NTU or less.

13. The composition according to claim 1, wherein the composition is a cosmetic composition.

14. A cosmetic process for treating a keratin substance, comprising:
applying the composition according to claim 1 to the keratin substance.
